(19) United States
(12) Patent Application Publication
(10) Pub. No.: US 2013/0073017 A1
(43) Pub. Date: Mar. 21, 2013

(54) Thermal vacuum therapy and apparatus thereof

(71) Applicants: Fong Yu Liu, Rowland Heights, CA (US); Timothy Samkit Tin, Rowland Heights, CA (US)

(72) Inventors: Fong Yu Liu, Rowland Heights, CA (US); Timothy Samkit Tin, Rowland Heights, CA (US)

(21) Appl. No.: 13/200,088

(22) Filed: Sep. 15, 2011

(51) Int. Cl. A61F 7/08

(52) U.S. Cl. 607/112

(57) ABSTRACT

An apparatus, for a thermal vacuum therapy by using a suction cup on a skin of a patient, includes a heating unit disposed in the suction cup. The heating unit includes a heat core made of high heat conductive material and a heat retention sleeve made of low heat conductive material, wherein the heat core is wrapped by the heat retention sleeve and is arranged in such a manner that after the heat core rapidly stores heat energy, the heat retention sleeve is adapted for keeping the heat energy at the heat core so as to enable the heat core being gradually released heat within the suction cup during cupping treatment.

### BACKGROUND OF THE PRESENT INVENTION

1. Field of Invention

The present invention relates to a medical treatment, an more particular to a thermal vacuum therapy especially fire cupping, wherein a heating unit is disposed in a suction cup for gradually releasing heat therewithin so as to prolong the heat stimulation of the acupuncture point and to mobilize blood within the suction area of the suction cup.

2. Description of Related Arts

Fire cupping is an ancient medical treatment to stimulate blood flow to certain areas of a patient's body. The technique of fire cupping is simple by creating a suction within a cup and applying the cup at the skin of the patient to mobilize blood flow in order to promote healing. Traditionally, the air within the cup is pre-heated before the cup is applied on the patient skin. As the air pressure rapidly reduces in the cup, a vacuum effect is created within the cup to pull the skin (especially the soft tissue thereat) up that the rim of the cup air-seals against the skin of the patient so as to create a local suction at the patient.

Moxibustion is another ancient medical treatment by placing a burnt moxa on an area, especially the acupuncture point, in order to gradually apply heat thereat to induce a gradual localized vasodilatation response.

Even though fire cupping and moxibustion have been prevalent in medical field for centuries, they both have several drawbacks. As it is mentioned above, the air within the cup must be pre-heated before the cup is applied on the skin of the patient. The air within the cup will be rapidly reduced in order to create the suction effect. Therefore, the traditional fire cupping can only create a local suction at the patient without any heat-treatment. On the other hand, skin damage, such as a burn mark on the skin of the patient may occur after moxibustion treatment. In particular, fire cupping and moxibustion cannot be used at the same time since it is impossible and is extremely dangerous to place the burnt moxa within the cup.

### SUMMARY OF THE PRESENT INVENTION

The invention is advantageous in that it provides a thermal vacuum therapy to promote treatment of fire cupping and moxibustion at the same time.

Another advantage of the invention is to provide a thermal vacuum therapy, especially fire cupping, wherein a heating unit is disposed in a suction cup for gradually releasing heat therewithin so as to prolong the heat stimulation of the acupuncture point and to mobilize blood within the suction area of the suction cup.

Another advantage of the invention is to a thermal vacuum therapy, wherein the heating unit can be rapidly heated up for storing heat energy and can be gradually release heat without directly burning the patient's skin.

Another advantage of the invention is to a thermal vacuum therapy, wherein the rim retainer has a flexible mouth adapted to seal at an irregular skin surface of the patient, such as a joint area especially at elbow, wrist, knee, and, ankle, that the conventional suction cup cannot be sealed thereat.

Another advantage of the invention is to a thermal vacuum therapy, wherein the heating unit can also emit far infrared during heat treatment and cupping treatment.

Another advantage of the invention is to a thermal vacuum therapy, wherein the apparatus of the present invention can be incorporated with any existing suction cup without alternating the original structure of the suction cup.

Another advantage of the invention is to a thermal vacuum therapy, wherein the heating unit can be properly and securely retained in the suction cup via the rim retainer and the length-adjustable sleeve.

Another advantage of the invention is to a thermal vacuum therapy, wherein the heating unit can be properly and securely retained in the suction cup via the unit holder within the suction cup. In addition, the unit holder has a plurality of ventilating ribs biased against the inner surface of the suction cup to ensure the suction effect therein.

Another advantage of the invention is to a thermal vacuum therapy, wherein medicine or herb can be administered through the skin of the patient by the heat from the heating unit during cupping treatment.

Another advantage of the invention is to a thermal vacuum therapy, wherein the temperature of the heating unit can be selectively adjusted to prevent the skin surface of the patient being burnt.

Another advantage of the invention is to a thermal vacuum therapy, wherein no expensive or complicated structure is required to employ in the present invention in order to achieve the above mentioned objects. Therefore, the present invention successfully provides an economic and efficient solution for providing the heat treatment at the same time during cupping treatment.

Additional advantages and features of the invention will become apparent from the description which follows, and may be realized by means of the instrumentalities and combinations particular point out in the appended claims.

According to the present invention, the foregoing and other objects and advantages are attained by an apparatus for a thermal vacuum therapy by using a suction cup on a skin of a patient, wherein the apparatus comprises a heating unit disposed in the suction cup. The heating unit comprises a heat core made of high heat conductive material and a heat retention sleeve made of low heat conductive material, wherein the heat core is wrapped by the heat retention sleeve and is arranged in such a manner that after the heat core rapidly stores heat energy, the heat retention sleeve is adapted for keeping the heat energy at the heat core so as to enable the heat core being gradually released heat within the suction cup during cupping treatment.

In accordance with another aspect of the invention, the present invention comprises a treatment method of cupping using a suction cup, comprising the steps of:

(a) pre-heating a heating unit at a predetermined temperature;

(b) disposing the heating unit in the suction cup at a position that a head of the heating unit is positioned toward an opening of the suction cup;

(c) coupling a rim retainer at a rim of the suction cup to retain the heating unit in the suction cup; and

(d) creating a suction effect within the suction cup for cupping treatment, wherein the heating unit is arranged for gradually releasing heat within the suction cup during cupping treatment.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is an exploded perspective view of an apparatus for a thermal vacuum therapy according to a preferred embodiment of the present invention.

FIG. 2 is a sectional view of the apparatus for the thermal vacuum therapy according to the above preferred embodiment of the present invention.

FIG. 3 illustrates an alternative mode of the apparatus for the thermal vacuum therapy according to the above preferred embodiment of the present invention.

FIG. 4 illustrates the heating units being preheated by the heat applier according to the above preferred embodiment of the present invention.

FIG. 5 is a sectional view of the apparatus for the thermal vacuum therapy according to a second preferred embodiment of the present invention.

FIG. 6 illustrates an alternative mode of the rim retainer of the apparatus for the thermal vacuum therapy according to the above preferred embodiment of the present invention.

### DETAILED DESCRIPTION OF THE PREFERRED EMBODIMENT

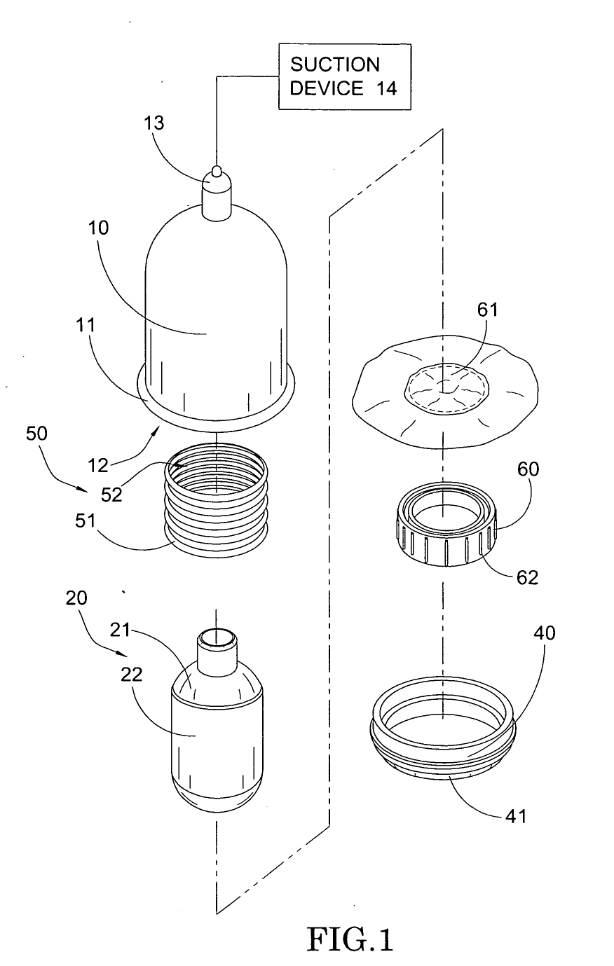

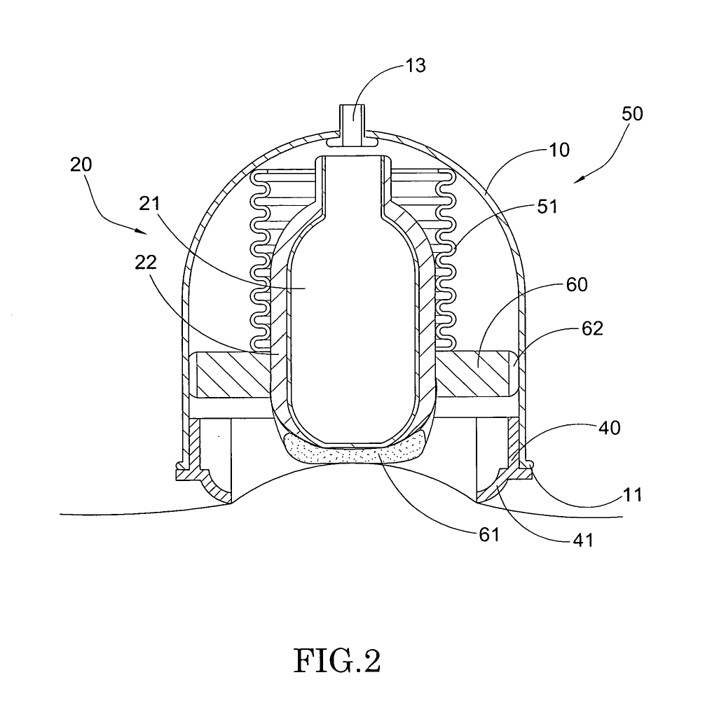

Referring to FIGS. 1 and 2 of the drawings, an apparatus for a thermal vacuum therapy according to a preferred embodiment of the present invention is illustrated, wherein the thermal vacuum therapy is a cupping therapy by using a suction cup 10 at the skin of the patient. Accordingly, the suction cup 10, such as an existing cup, has a rim 11 defining an opening 12, wherein an air valve 13 is provided at the ceiling of the suction cup 10 to create a suction effect via a suction device 14 such as a manual suction pump or an electric suction pump.

According to the preferred embodiment, the apparatus comprises a heating unit 20 disposed in the suction cup 10, wherein the heating unit 20 comprises a heat core 21 made of high heat conductive material and a heat retention sleeve 22 made of low heat conductive material. The heating unit 20 is a non-battery powered unit and a non electrical conducted unit. Therefore, it is safe to use the heating unit 20 within the suction cup 10 without any electrical wire connection or charging wire connection for safety purpose. The heating unit 20 is pre-heated to store heat energy.

The heat core 21 is wrapped by the heat retention sleeve 22 and is arranged in such a manner that after the heat core 21 rapidly stores heat energy, the heat retention sleeve 22 is adapted for keeping the heat energy at the heat core 21 so as to enable the heat core 21 being gradually released heat within the suction cup 10 during cupping treatment. Preferably, the heat core 21 is made of copper and the heat retention sleeve 22 is made of ceramics. Alternatively, the heat core 21 can be made of brass and aluminum that the head of the heat core 21 is made of brass for heat conduction and the body of the heat core 21 is made of aluminum for reducing the overall weight of the head core 21. Preferably, the heat retention sleeve 22 is also adapted for emitting far infrared at the same time when the heat is dissipated from the heat core 21.

As shown in FIG. 2, the head of the heating unit 20 is positioned toward the opening 12 of the suction cup 10 and a tail of the heating unit 20 is positioned toward the ceiling of the suction cup 10 when the heating unit 20 is disposed in the suction cup 10.

The head of the heating unit 20 preferably has a spherical shape for enlarging the surface area thereof in order to dissipate the heat toward the skin of the patient.

Preferably, the heat core 21 has a cylindrical shape defining a cylindrical body, a spherical head portion and an elongated tail portion. In addition, the heat core 21 has a flat contacting surface formed at the head portion for increasing the contact area thereof.

In order to enhance the heat treatment for the patient, the head portion of the heat core 21 is unwrapped by the heat retention sleeve 22, wherein the head portion of the heat core 21 is defined at the head of the heating unit 20. Therefore, the heat at the head of the heat core 21 can effectively dissipate toward the skin of the patient.

In addition, the inner side of the tail portion of the heat core 21 is also unwrapped by the heat retention sleeve 22 to define a heat charging portion of the heating unit 20 for an external heat applying to the heat core 21. In other words, the heat retention sleeve 22 wraps around the outer side of the heat core 21, especially the cylindrical body thereof, while the head portion of the heat core 21 is exposed in an unwrapped manner. In particular, the tail portion of the heat core 21 has an open end adapted for being pre-heated thereat in order to heat up the heat core 21. It is worth mentioning that the heat core 21 will be pre-heated at a predetermined temperature that even though the head portion of the heat core 21 will contact with the skin of the patient, the skin thereof will not be burnt by the heat core 21, so as to prevent any skin damage by the heating unit 20.

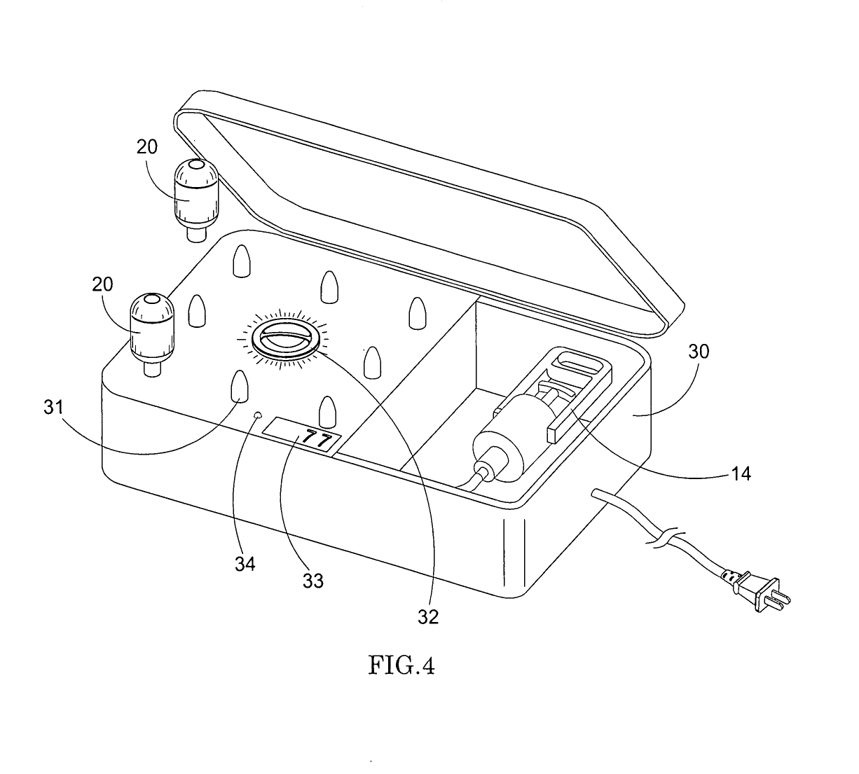

In order to pre-heat the heating unit 20, the apparatus further comprises a heat applier 30 for applying the external heat to the heat core, as shown in FIG. 4. The heat applier 30 comprises one or more heat generators 31 detachably coupled with one or more heating units 20 in order to pre-heat the heating units 20 at the same time. As shown in FIG. 4, each of the heat generators 31 forms in an elongated shape and extends vertically, wherein when the heat applier 30 is electrically switched on, each of the heat generators 31 will generates heat therealong. Accordingly, the tail portion of the heat core 21, i.e. the heat charging portion of the heating unit 20, forms an elongated tubular configuration that the free end of the heat generator 31 is adapted to slidably insert into the tail portion of the heat core 21 into the body thereof. Therefore, the heat core 21 will heat-conduct with the heat generator 31 in order to pre-heat the heat core 21. Therefore, after the heating unit 20 is pre-heated, the heating unit 20 is adapted for disposing in the suction cup 10. It is appreciated that the heat applier 30 is incorporated with a temperature control for selectively controlling the temperature of the heat generator 31 in order to pre-heat the heating unit 20.

The heat applier 30 further comprises a heat adjuster 32 operatively linked to the heat generators 31 to selectively adjust the temperature thereof. Accordingly, the heat adjuster 32 is operatively connected to the heat generators 31 through a heat control circuit. Accordingly, the heat control circuit is also a maximum temperature control for controlling the maximum temperature of the heat generators 31 so as to prevent the heat generators 31 being overheated. Preferably, the temperature of each of the heat generators 31 has a range between 20° C. to 80° C. It is worth mentioning that the heat control circuit is integrated with a fuse circuit for overcurrent protection of the heat generators 31.

The heat applier 30 further comprises a temperature indicator 33 and an operation indicator 34 operatively linked to the heat generators 31. Accordingly, the temperature indicator 33 comprises a thermometer unit and a display to indicate the temperature of the heat generators 31. The operation indicator 34 is a light indicator and is arranged in such a manner that when the heat generators 31 are powered on, the light indicator will generate light to indicate the “On” status thereof. When the heat generators 31 reach the preset temperature via the heat adjuster 32, the light from the light indicator will be automatically turned off to indicate the “Off” status thereof.

As shown in FIGS. 1 and 2, the apparatus further comprises a rim retainer 40 for detachably coupling with the rim 11 of the suction cup 10 to retain the heating unit 20 therewith. The rim retainer 40 is made of sealing material and has a ring-shape adapted for sealedly coupling with the rim 11 of the suction cup 10, wherein an inner diameter of the rim retainer 40 is smaller than the diameter of the rim 11 of the suction cup 10. Accordingly, the inner diameter of the rim retainer 40 can be configured to be smaller a diameter of the heating unit 20 for retaining the heating unit 20 within the suction cup 10. In other words, when the rim retainer 40 is coupled at the rim 11 of the suction cup 10, the heating unit 20 is seated at the rim retainer 40 at the inner diameter thereof within the suction cup 10. It is worth mentioning that the rim retainer 40 forms a replacement of the rim 11 of the suction cup 10 to apply to the skin of the patient.

According to the preferred embodiment, the rim retainer 40 is made of soft rubber having an outer diameter corresponding to the diameter of the rim 11 of the suction cup 10, wherein the rim retainer 40 is coupled at the inner side of the rim 11 of the suction cup 10 in an air-seal manner so as to retain the rim retainer 40 around the opening 12 of the suction cup 10. Preferably, the rim retainer 40 is partially coupled at the rim 11 of the suction cup 10 that an upper portion of the rim retainer 40 is coupled at the inner side of the rim 11 of the suction cup 10 while a lower portion of the rim retainer 40 is extended out of the rim 11 of the suction cup 10. As the conventional cupping treatment, a cupping mark will created on the skin of the patient by the rim 11 of the suction cup 10. In addition, some patients will feel uncomfortable by the rigidly of the rim 11 of the suction cup 10. Therefore, during the cupping operation of using the rim retainer 40, the lower portion of the rim retainer 40 will contact with the skin of the patient to minimize the uncomfortable feeling of the rim 11 of the suction cup 10. Since the rim retainer 40 is made of soft and sealing material, the rim retainer 40 can fit to seal on the skin of the patient so as to enhance the suction effect within the suction cup 10. By selecting different sizes of the inner diameter of the rim retainer 40, the suction 10 can be used as one larger size to selectively apply to different areas of the patient's skin.

It is worth mentioning that the inner diameter of the rim retainer 40 can be selected to adjust the size of the rim retainer 40 being applied at the patient's skin. When a larger inner diameter of the rim retainer 40 is selected, the rim retainer 40 can be applied on a larger skin surface of the patient, such as the back of the patient. When a smaller inner diameter of the rim retainer 40 is selected, the rim retainer 40 can be applied on a smaller skin surface of the patient, such as the elbow, wrist, knee, and, ankle of the patient. It is appreciated that the position of the heating unit 20 can be selectively adjusted through the inner diameter of the rim retainer 40. When a larger inner diameter of the rim retainer 40 is selected, the heating unit 20 will be dropped down within the suction cup 10 that the head of heating unit 20 will be closer to the skin of the patient. When a smaller inner diameter of the rim retainer 40 is selected, the heating unit 20 will be lifted up within the suction cup 10 that the head of heating unit 20 will be away from the skin of the patient for preventing said heating unit being directly contacting with the skin of the patient during cupping treatment.

In particular, the rim retainer 40 has a flexible mouth 41 defined at the lower portion of the rim retainer 40 to contact with the skin of the patient. Accordingly, the flexible mouth 41 has a tapering shape that an outer diameter of the flexible mouth 41 is gradually reducing toward a center thereof. Therefore, the flexible mouth 41 can tightly seal at relatively small surface area of the patient during cupping treatment. It is worth mentioning that the conventional suction cup can only seal at the relatively large regular skin surface of the patient such as the back of the patient. The flexible mouth 41 of the present invention enables the suction cup to seal at an irregular skin surface of the patient, such as a joint area especially at elbow, wrist, knee, and, ankle, that the conventional suction cup cannot be sealed thereat.

The apparatus of the present invention further comprises a length-adjustable sleeve 50 for adjusting a position of the heating unit 20 within the suction cup 10. Accordingly, when the heating unit 20 is disposed in the suction cup 10, the heating unit 20 may be unavoidably moved around inside the suction cup 10, such that the head of the heating unit 20 cannot be ensured to point downwardly toward the skin of the patient. The length-adjustable sleeve 50 is disposed in the suction cup 10 to ensure the correct position of the heating unit 20 within the suction cup 10.

As shown in FIG. 2, the length-adjustable sleeve 50 has a zigzag surrounding wall 51 biasing against the heating unit 20, wherein the length-adjustable sleeve 50 is arranged for biasing a top ceiling of the suction cup 10 to push the heating unit 20 towards the opening 12 of the suction cup 10 when the heating unit 20 is disposed in the suction cup 10. Preferably, the length-adjustable sleeve 50 is made of flexible material that the surrounding wall 51 can be extended or squeezed to selectively adjust the length thereof.

According to the preferred embodiment, the length-adjustable sleeve 50 has a tubular structure defining a tubular cavity 52 within the surrounding wall 51, wherein the heating unit 20 is received in the cavity 52 of the length-adjustable sleeve 50 at the bottom portion thereof. Preferably, the head of the heating unit 20 is protruded from the bottom end of the length-adjustable sleeve 50. The top end of the length-adjustable sleeve 50 is arranged to bias against the top ceiling of the suction cup 10 such that the surrounding wall 51 will be squeezed to self-adjust the length of the length-adjustable sleeve 50 between the top and bottom ends thereof. It is worth mentioning that the heating unit 20 is retained at a position that the head of the heating unit 20 will always point downward toward the opening 12 of the suction cup 10 via the length-adjustable sleeve 50.

As heat is continuously released by the heating unit 20 within the suction cup 10, the treatment element can also be disposed in the suction cup 10 to effectively administer the treatment element to the patient through the skin thereof during the heat treatment. It is worth mentioning that the heating unit 20 is positioned between the length-adjustable sleeve 50 and the rim retainer 40 such that when the heating unit 20 is pushed to the opening 12 of the suction cup 10 via the length-adjustable sleeve 50, the head of the heating unit 20 is retained and seated at the rim retainer 40 for preventing the heating unit 20 being pushed out of the suction cup 10 as shown in FIG. 2.

The apparatus of the present invention further comprises a treatment holder 60 for replaceably holding a treatment pack 61 to administer the treatment element therein to the patient. The treatment holder 60 is disposed at the suction cup 10 around the rim 11 thereof, wherein when the treatment pack 61 is held by the treatment holder 60, the treatment element in the treatment pack 61 can be effectively administered to the patient through the heat treatment. Accordingly, the treatment pack 61 contains medicine or herbs as the treatment element that when heat is applied at the treatment pack 61, the treatment element in the treatment pack 61 can be effectively administered to the patient.

As shown in FIG. 2, the treatment holder 60, having a ring shape, is coupled at the heating unit 20 around the head thereof for enabling the treatment pack 61 being contacted with the head of the heating unit 20 so as to enable the treatment element in the treatment pack 61 being administered through the skin of the patient by the heat from the heating unit 20.

In particular, the treatment holder 60 is arranged to seal at the inner surface of the suction cup 10 to retain the treatment pack 61 in position, wherein the treatment pack 61 is held between the head of the heating unit 20 and the skin of the patient, such that the heat dissipated from the heating unit 20 will be applied on the skin of the patient through the treatment pack 61 to ensure the treatment element being effectively administered to the patient. It is worth mentioning that when the treatment pack 61 is held within the suction cup 10 via the treatment holder 60, the treatment pack 61 is protruded out at the head of the heat core 21 to contact with the skin of the patient. The treatment element can be selected to be packed in the treatment pack 61 according to the need of the patient.

In addition, alcohol, such as ethanol, is applied at the treatment pack 61 being the suction cup 10 being applied on the skin of the patient. Accordingly, alcohol is used as antiseptic and is volatile that when alcohol is applied on the skin of the patient, the pores of the skin will be expanded. Preferably, alcohol can be sprayed at the treatment pack 61 before the treatment pack 61 is contacted with the patient' skin. Therefore, the treatment element can be effectively administered through the skin of the patient. In addition, when the alcohol is applied, the temperature of the heat core 21 can be lowered without reducing the effectiveness of the heat treatment.

According to the preferred embodiment, the apparatus further comprises a ventilating means for ensuring the suction effect being created around the head of the heating unit 20 during cupping treatment. In particular, the ventilating means will provide at one or more of different components for ensuring the ventilation within the suction cup 10.

The treatment holder 60 has a plurality of ventilating ribs 62, as the ventilating means, spacedly formed at the outer surface thereof for biasing against the inner surface of the suction cup 10 to ensure the suction effect therewithin, especially around the head of the heating unit 20. Therefore, the ventilating ribs 62 will prevent the outer surface of the treatment holder 60 being directly and sealedly contacted with the inner surface of the suction cup 10 which may reduce the suction effect thereof. It is appreciated that the treatment holder 60 can also retained and seated at the rim retainer 40.

In order to use the apparatus of the present invention, the user, such as a physician or an operator, can pre-heat the heating units 20 via the heat applier 30 by placing the heat charging portions of the heating units 20 at the heat generators 31 respectively so as to enable the heating units 20 being heated up at the same time. The heating unit 20 can be placed in the suction cup 10 at a position that the head of the heating unit 20 is positioned toward the opening 12 of the suction cup 10 and the tail of the heating unit 20 is positioned toward the ceiling of the suction cup 10. It is worth mentioning that the length-adjustable sleeve 50 can be pre-set to the heating unit 20 that the heating unit 20 is already placed in the length-adjustable sleeve 50 such that when the heating unit 20 is disposed in the suction cup 10, the top end of the length-adjustable sleeve 50 is biased against the ceiling of the suction cup 10. It is appreciated that the heating unit 20 can be placed in the length-adjustable sleeve 50 after the heating unit 20 is pre-heated. Then, the treatment pack 61 can be placed at the treatment holder 60 before the treatment holder 60 is coupled at the heating unit 20 to ensure the head of the heating unit 20 contacts with the treatment pack 61. The rim retainer 40 can be coupled at the rim 11 of the suction cup 10 to ensure the heating unit 20 being properly retained in the suction cup 10. It is worth mentioning that the treatment holder 60 is optionally placed in the suction cup 10 unless the patient is required for the treatment element to be administered. Once the apparatus is properly setup, the suction cup 10 can be placed on the skin of the patient. Once the suction device 14 is operated, the suction effect is created within the suction cup 10 for cupping treatment.

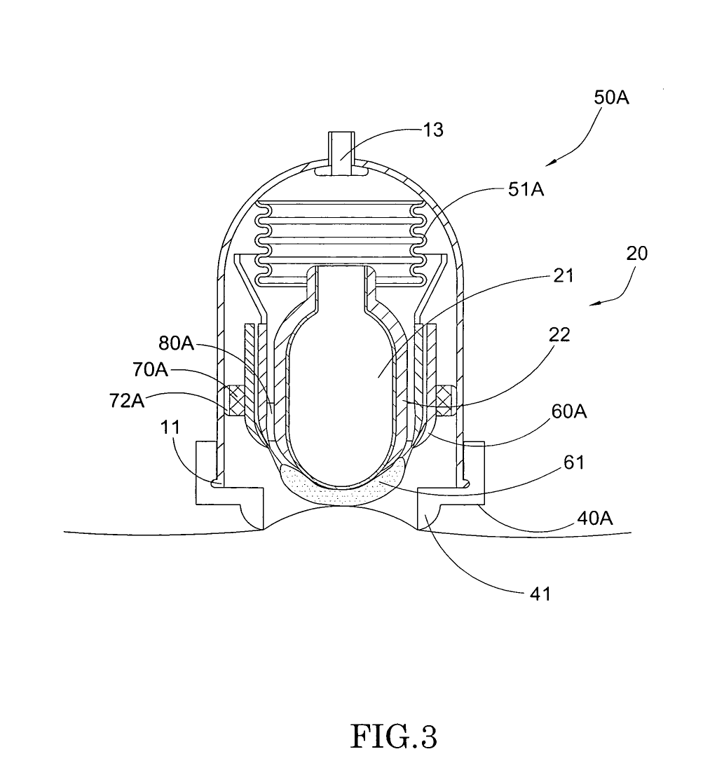

FIG. 3 illustrates an alternative mode of the preferred embodiment, wherein the rim retainer 40A for detachably coupling with the rim 11 of the suction cup 10 to retain the heating unit 20 therewith. The rim retainer 40A is made of sealing material and has a ring-shape adapted for sealedly coupling with the rim 11 of the suction cup 10, wherein an inner diameter of the rim retainer 40A is smaller than a diameter of the rim 11 of the suction cup 10.

As shown in FIG. 3, the rim retainer 40A is made of soft rubber having an outer diameter corresponding to the diameter of the rim 11 of the suction cup 10, wherein the rim retainer 40A is coupled at the outer side of the rim 11 of the suction cup 10 in an air-seal manner so as to retain the rim retainer 40A around the opening 12 of the suction cup 10. Therefore, during the cupping operation of using the rim retainer 40A, the rim retainer 40A will contact with the skin of the patient to minimize the uncomfortable feeling of the rim 11 of the suction cup 10. Since the rim retainer 40A is made of soft and sealing material, the rim retainer 40A can fit to seal on the skin of the patient so as to enhance the suction effect within the suction cup 10.

In addition, the length-adjustable sleeve 50A has a zigzag surrounding wall 51A biasing against the heating unit 20, wherein the length-adjustable sleeve 50A has a tubular structure that the bottom end of the surrounding wall 51A is biased against the tail of the heating unit 20 while the top end of the surrounding wall 51A is biased against the ceiling of the suction cup 10. In other words, the length-adjustable sleeve 50A is positioned between the heating unit 20 and the ceiling of the suction cup 10 for adjusting the position of the heating unit 20 within the suction cup 10.

As shown in FIG. 3, the treatment holder 60A, having a cup shape, is coupled at the heating unit 20 around the body thereof, wherein the treatment holder 60A has a bottom opening for retaining the treatment pack 61 thereat. Therefore, when the treatment holder 60A is coupled at the heating unit 20, the treatment holder 60A is adapted for enabling the treatment pack 61 being contacted with the head of the heating unit 20 so as to enable the treatment element in the treatment pack 61 being administered through the skin of the patient by the heat from the heating unit 20.

As shown in FIG. 3, the apparatus further comprises a unit holder 70A which is embodied as the treatment holder 60 of the above embodiment, wherein the unit holder 70A is formed as a holding device to retain the heating unit 20 in position without holding the treatment pack 61. In other words, the treatment pack 61 is held by the treatment holder 60A in this embodiment.

Likewise, the unit holder 70A, having a ring shape, is coupled at the heating unit 20 around the head thereof for enabling the treatment pack 61 being contacted with the head of the heating unit 20 so as to retain the heating unit 20 in position.

In particular, the unit holder 70A is arranged to seal at the inner surface of the suction cup 10 to retain the heating unit 20 in position, wherein the unit holder 70A has a plurality of ventilating ribs 72A, as the ventilating means, spacedly formed at the outer surface thereof for biasing against the inner surface of the suction cup 10 to ensure the suction effect therewithin. Therefore, the ventilating ribs 72 will prevent the outer surface of the unit holder 70A being directly and sealedly contacted with the inner surface of the suction cup 10 which may reduce the suction effect thereof.

As shown in FIG. 3, the apparatus further comprises a heat dissipating element 80A mounted around the heating unit 20 for evenly dissipating heat around the head thereof. The heat dissipating element 80A has a ring shape and is made of heat insulation material, wherein when the heat is dissipated by the heating unit 20, the heat dissipating element 80A will ensure the dissipating direction of heat toward the head of the heating unit 20.

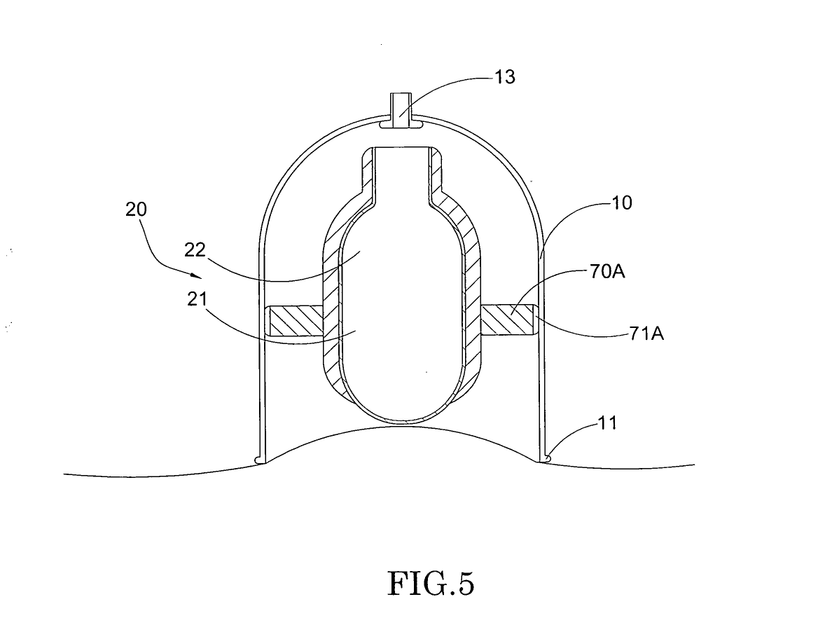

As it is mentioned above, the heat core 21 will be pre-heated at a predetermined temperature that the skin of the patient will not be burnt or damaged by directly contacting the head portion of the heat core 21 with the skin of the patient. Therefore, the user can directly dispose the heating unit 20 in the suction cup 10 and can couple the rim retainer 40 to the rim 11 of the suction cup 10 to retain the heating unit 20 in the suction cup 10 without any length-adjustable sleeve 50 or treatment holder 60, as shown in FIG. 5. Preferably, the heating unit 20 is retained within the suction cup 10 by the unit holder 70A.

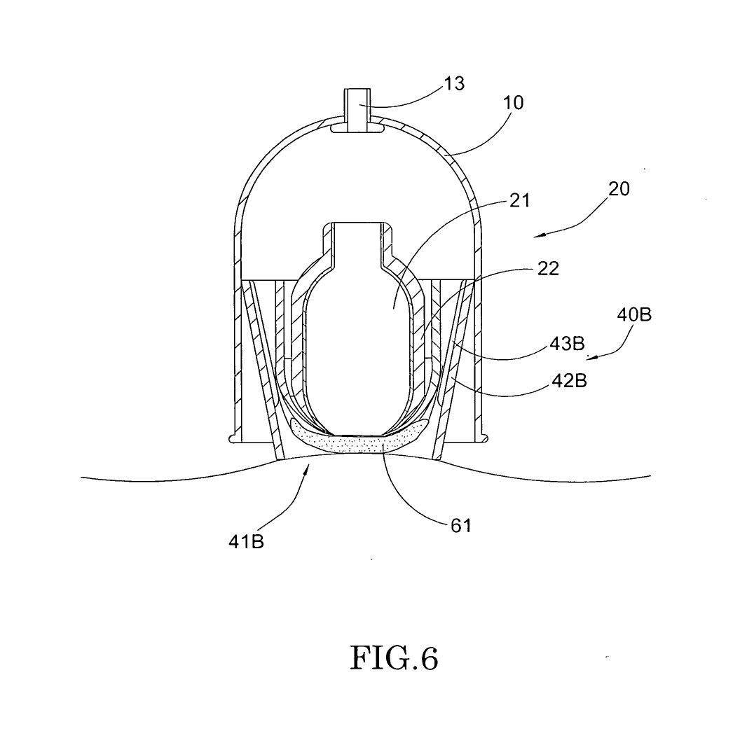

FIG. 6 further illustrates another alternative mode of the rim retainer 40B, wherein the rim retainer 40B provides multi-functions for the heating unit 20. As shown in FIG. 6, the rim retainer 40B has a conical retaining body 42B, which is made of soft material such as rubber, defining an upper opening and a lower opening which has a diameter smaller than a diameter of the upper opening. The flexible mouth 41B is defined at the lower opening of the retaining body 42B. The conical retaining body 42B is arranged to couple with and seal at the suction cup 10 at a position that the upper opening of the retaining body 42B is located within the suction cup 10 to seal at the inner side of suction cup 10 while the lower opening of the retaining body 42B is protruded out of the rim 11 of the suction cup 10.

The heating unit 20 is disposed within and held by the retaining body 42B to retain the heating unit 20 within the suction cup 10. In particular, the rim retainer 40B further has a plurality of ventilating ribs 43B, as the ventilating means, spacedly formed at the inner wall thereof for biasing the heating unit 20 to ensure the suction effect within the suction cup 10. The ventilating ribs 43B are integrally extended from the upper opening of the retaining body 42B toward the lower opening thereof. Therefore, the ventilating ribs 43B will prevent the outer surface of heating unit 20 being directly and sealedly contacted with the inner wall of the retaining body 42B which may reduce the suction effect thereof.

In addition, the retaining body 42B further forms as the treatment holder to retain the treatment pack 61 in position. In other words, when the heating unit 20 is disposed within the retaining body 42B, the treatment pack 61 is also held within the retaining body 42B at the head of the heating unit 20, as shown in FIG. 6.

As it is mentioned above, when a bigger size of the suction cup 10 is used, a relatively stronger suction effect will be created therewithin. However, the bigger suction cup 10 cannot be used to apply at the irregular skin surface of the patient, such as a joint area especially at elbow, wrist, knee, and, ankle. Therefore, a smaller size of the suction cup 10 must be used. On the other hand, the smaller suction cup 10 will provide weaker suction effect thereof.

Since the flexible mouth 41B of the retaining body 42B is smaller than the rim of the suction cup 10, the flexible mouth 41B of the retaining body 42B enables the suction cup 10 to seal at the irregular skin surface of the patient, such as a joint area especially at elbow, wrist, knee, and, ankle, that the conventional suction cup cannot be sealed thereat. However, a bigger suction cup 10 can be used to incorporate with the retaining body 42B. Therefore, the bigger suction cup 10 will provide stronger suction effect while the retaining body 42B will be applied at the irregular skin surface of the patient.

According to the preferred embodiment, the heating unit 20 functions as a burnt moxa placed on an area, especially the acupuncture point, as the technique of Moxibustion in order to gradually apply heat thereat to induce a gradual localized vasodilatation response. However, no burn mark will be created on the skin of the patient by using the heating unit 20.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. An apparatus for a thermal vacuum therapy by using a suction cup on a skin of a patient, comprising:
a heating unit, which is arranged for being disposed in said suction cup, comprising a heat core made of high heat conductive material and a heat retention sleeve made of low heat conductive material, wherein said heat core is wrapped by said heat retention sleeve and is arranged in such a manner that after said heat core rapidly stores heat energy, said heat retention sleeve is adapted for keeping said heat energy at said heat core so as to enable said heat core being gradually released heat within said suction cup during cupping treatment.

2. The apparatus, as recited in claim 1, wherein an inner side of a tail portion of said heat core is unwrapped by said heat retention sleeve to define a heat charging portion of said heating unit for an external heat applying to said heat core.

3. The apparatus, as recited in claim 1, further comprising a rim retainer for detachably coupling with a rim of said suction cup, wherein said rim retainer is made of sealing material and has a ring-shape adapted for sealedly coupling with said rim of said suction cup, wherein an inner diameter of said rim retainer is smaller than a diameter of said rim of said suction cup.

4. The apparatus, as recited in claim 2, further comprising a rim retainer for detachably coupling with a rim of said suction cup, wherein said rim retainer is made of sealing material and has a ring-shape adapted for sealedly coupling with said rim of said suction cup, wherein an inner diameter of said rim retainer is smaller than a diameter of said rim of said suction cup.

5. The apparatus, as recited in claim 1, wherein a head portion of said heat core is unwrapped by said heat retention sleeve.

6. The apparatus, as recited in claim 2, wherein a head portion of said heat core is unwrapped by said heat retention sleeve.

7. The apparatus, as recited in claim 4, wherein a head portion of said heat core is unwrapped by said heat retention sleeve.

8. The apparatus, as recited in claim 1, further comprising a length-adjustable sleeve for adjusting a position of said heating unit within said suction cup, wherein said length-adjustable sleeve has a zigzag surrounding wall biasing against said heating unit, wherein said length-adjustable sleeve is arranged for biasing a top ceiling of said suction cup to push said heating unit towards an opening of said suction cup when said heating unit is disposed in said suction cup.

9. The apparatus, as recited in claim 7, further comprising a length-adjustable sleeve for adjusting a position of said heating unit within said suction cup, wherein said length-adjustable sleeve has a zigzag surrounding wall biasing against said heating unit, wherein said length-adjustable sleeve is arranged for biasing a top ceiling of said suction cup to push said heating unit towards an opening of said suction cup when said heating unit is disposed in said suction cup.

10. The apparatus, as recited in claim 1, further comprising a treatment holder for replaceably holding a treatment pack to administer treatment element therein to the patient, wherein said treatment holder is coupled at said heating unit for enabling said treatment pack being contacted with said heating unit so as to enable said treatment element in said treatment pack being administered through the skin of the patient by the heat from said heating unit.

11. The apparatus, as recited in claim 9, further comprising a treatment holder for replaceably holding a treatment pack to administer treatment element therein to the patient, wherein said treatment holder is coupled at said heating unit for enabling said treatment pack being contacted with said heating unit so as to enable said treatment element in said treatment pack being administered through the skin of the patient by the heat from said heating unit.

12. The apparatus, as recited in claim 10, wherein said treatment holder further comprises a plurality of ventilating ribs spacedly formed thereat for ensuring the suction effect being created around a head of said heating unit.

13. The apparatus, as recited in claim 11, wherein said treatment holder further comprises a plurality of ventilating ribs spacedly formed thereat for ensuring the suction effect being created around a head of said heating unit.

14. The apparatus, as recited in claim 1, wherein said heat core is made of a material selected from the group consisting of copper, brass, and aluminum, wherein said heat retention sleeve is made of ceramics.

15. The apparatus, as recited in claim 13, wherein said heat core is made of a material selected from the group consisting of copper, brass, and aluminum, wherein said heat retention sleeve is made of ceramics.

16. The apparatus, as recited in claim 2, further comprising a heat applier for applying said external heat to said heat core, wherein said heat applier comprises a heat generator detachably coupled with said heat charging portion of said heating unit to pre-heat said heat core, such that after said heating unit is pre-heated, said heating unit is adapted for disposing in said suction cup.

17. The apparatus, as recited in claim 15, further comprising a heat applier for applying said external heat to said heat core, wherein said heat applier comprises a heat generator detachably coupled with said heat charging portion of said heating unit to pre-heat said heat core, such that after said heating unit is pre-heated, said heating unit is adapted for disposing in said suction cup.

18. A treatment method of cupping using a suction cup, comprising the steps of:
(a) disposing a heating unit in said suction cup at a position that a head of said heating unit is positioned toward an opening of said suction cup, wherein said heating unit is a non-battery powered unit for heat generation; and
(b) creating a suction effect within said suction cup for cupping treatment, wherein said heating unit is arranged for gradually releasing heat within said suction cup during cupping treatment.

19. The method, as recited in claim 18, wherein said heating unit comprises a heat core made of high heat conductive material and a heat retention sleeve made of low heat conductive material, wherein said heat core is wrapped by said heat retention sleeve and is arranged in such a manner that after said heat core rapidly stores heat energy, said heat retention sleeve is adapted for keeping said heat energy at said heat core so as to enable said heat core being gradually released heat within said suction cup during cupping treatment.

20. The method, as recited in claim 19, before the step (b), further comprising a step of detachably coupling a rim retainer with a rim of said suction cup to retain said heating unit in said suction cup, wherein an inner diameter of said rim retainer is smaller than a diameter of said heating unit for retaining said heating unit within said suction cup.

21. The method, as recited in claim 20, before the step (b), further comprising a step of placing a length-adjustable sleeve within said suction cup for adjusting a position of said heating unit within said suction cup, wherein said length-adjustable sleeve is arranged for biasing a top ceiling of said suction cup to push said heating unit towards said opening of said suction cup when said heating unit is disposed in said suction cup.

22. The method, as recited in claim 20, before the step (b), further comprising a step of placing a treatment holder at said heating unit for replaceably holding a treatment pack thereat, wherein said treatment pack is held to be contacted with said heating unit so as to enable treatment element in said treatment pack being administered through the skin of the patient by the heat from said heating unit.

23. The method, as recited in claim 18, before the step (a), further comprising a step of pre-heating said heating unit at a predetermined temperature before said heating unit is disposed in said suction cup.

24. The method, as recited in claim 22, before the step (a), further comprising a step of pre-heating said heating unit at a predetermined temperature before said heating unit is disposed in said suction cup.

25. The method, as recited in claim 18, wherein said heat core is made of a material selected from the group consisting of copper, brass, and aluminum, wherein said heat retention sleeve is made of ceramics.

26. The method, as recited in claim 24, wherein said heat core is made of a material selected from the group consisting of copper, brass, and aluminum, wherein said heat retention sleeve is made of ceramics.
